(19) United States
(12) Patent Application Publication
(10) Pub. No.: US 2009/0118515 A1
(43) Pub. Date: May 7, 2009

(54) PROCESS FOR PREPARING INTERMEDIATES USEFUL TO PREPARE CERTAIN ANTIBACTERIAL N-FORMYL HYDROXYLAMINES

(75) Inventors: Joel SLADE, Flanders, NJ (US); James Anthony VIVELO, Basking Ridge, NJ (US); Guang-Pei CHEN, Livingston, NJ (US); Joginder Singh BAJWA, Elmwood Park, NJ (US); David John PARKER, Livingston, NJ (US)

Correspondence Address: LAHIVE & COCKFIELD, LLP;FLOOR 30, SUITE 3000, ONE POST OFFICE SQUARE, BOSTON, MA, 02109, US

(73) Assignee: NOVARTIS AG, Basel (CH)

(21) Appl. No.: 12/263,992

(22) Filed: Nov. 3, 2008

Related U.S. Application Data
(63) Continuation of application No. 10/561,754, filed on May 19, 2006.
(60) Provisional application No. 60/482,686, filed on Jun. 26, 2003.

(51) Int. Cl. C07D 401/12; C07D 263/20; C07C 51/373

(52) U.S. Cl. 546/279.1; 548/230; 562/567

(57) ABSTRACT

The present invention is directed to a process for preparing intermediates that are useful to prepare certain antibacterial N-formyl hydroxylamine compounds which are peptide deformylase inhibitors.

This invention is directed to a process for preparing intermediates that are useful to prepare certain antibacterial N-formyl hydroxylamine compounds.

Peptide deformylase is a metallopeptidase found in prokaryotic organisms such as bacteria. Protein synthesis in prokaryotic organisms begins with N-formyl methionine (fMet). After initiation of protein synthesis, the formyl group is removed by the enzyme peptide deformylase (PDF); this activity is essential for maturation of proteins. It has been shown that PDF is required for bacterial growth. See Chang et al., J. Bacteriol., Vol. 171, pp. 4071-4072 (1989); Meinnel et al., J. Bacteriol., Vol. 176, No. 23, pp. 7387-7390 (1994); Mazel et al., EMBO J., Vol. 13, No. 4, pp. 914-923 (1994). Since protein synthesis in eukaryotic organisms does not depend on fMet for initiation, agents that will inhibit PDF are attractive candidates for development of new anti-microbial and anti-bacterial drugs.

Co-pending application Ser. No. 10/171,706, filed Jun. 14, 2002 (incorporated herein by reference in its entirety), PCT equivalent published as WO 02/102790 A1, discloses novel N-formyl hydroxylamine compounds that inhibit PDF and are therefore useful as antibacterial agents. The compounds disclosed therein are certain N-[1-oxo-2-alkyl-3-(N-hydroxyformamido)-propyl]-(carbonylamino-aryl or -heteroaryl)-azacyclo₄₋₇alkanes or thiazacyclo₄₋₇alkanes which are described in more detail hereinafter. An improved process has been discovered for preparing intermediates useful for preparing these N-[1-oxo-2-alkyl-3-(N-hydroxyformamido)-propyl]-(carbonylamino-aryl or -heteroaryl)-azacyclo₄₋₇alkanes or thiazacyclo₄₋₇alkanes.

The present invention is directed to a novel process for preparing certain intermediates which are useful to prepare certain N-formyl hydroxylamine compounds which are useful for inhibiting bacteria.

More specifically, the present invention is directed to a process for preparing a compound of the formula (VII)

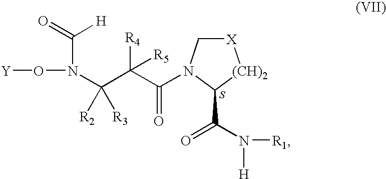

comprising Step 1A:

Contacting a compound of the formula (I)

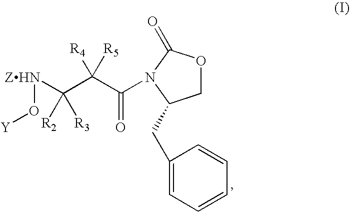

with a base in a suitable solvent to form the free base of compound (I), i.e., compound (II) of the formula (II)

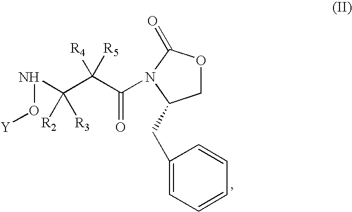

followed by Step 1B.

Contacting compound (II) with a strong nucleophile/weak base in a suitable solvent under conditions to form compound (III) of the formula (III)

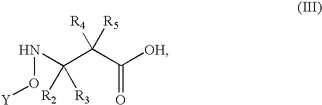

followed by Step 2A:

Contacting compound (III) with a formulating agent in a suitable solvent under conditions suitable to form a compound of formula (IV)

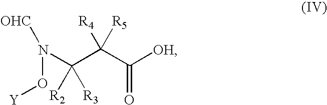

followed by Step 2B:

Contacting compound (IV) with an amine or an alkaline metal hydroxide in a suitable solvent under conditions to form a compound of formula (V)

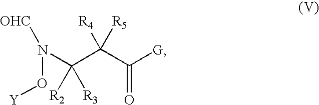

followed by Step 3:

Contacting compound (V) with a compound of formula (VI)

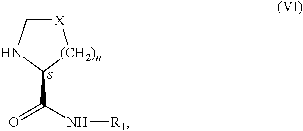

in the presence of a suitable base and one or more coupling agents in a suitable solvent under conditions to form a compound of formula (VII)
wherein
Y is a hydroxy protecting group;
Each of R₂, R₃, R₄ and R₅ is, independently, hydrogen or alkyl, or (R₂ and R₃) and/or (R₄ and R₅) collectively form a C₄₋₇cycloalkyl;
G is —O^{⊖}metal^{⊕} or —OH.amine;
X is —CH₂—, —S—, —CH(OH)—, —CH(OR)—, —CH(SH)—, —CH(SR)—, —CF₂—, —C═N(OR)— or —CH(F)—;
wherein
   R is alkyl;
   R₁ is aryl or heteroaryl;
   Z is a strong organic or inorganic acid; and
   n is 0-3, provided that when n is 0, X is —CH₂—.

When the desired product is an N-oxide of an aromatic moiety having a nitrogen heteroatom (e.g., when R₁ is Formula X, Xa or Xb), typically a pyridine derivative, it is necessary to perform an additional step after step 3, i.e., to oxidize the N of the aromatic ring (Step 4). Therefore, the present invention includes Step 4 which comprises contacting the compound of formula VII, wherein R₁ is heteroaryl having an N heteroatom, with an oxidizing agent to form the corresponding N-oxide derivative.

In addition to the above process comprising Steps 1A-4 the present invention is directed to each of the steps individually, and to any two or more sequential steps.

In particular, the present invention provides a process for preparing intermediates useful in the preparation of a N-[1-oxo-2-alkyl-3-(N-hydroxyformamido)-propyl]-(carbonylamino-aryl or -heteroaryl)-azacyclo₄₋₇alkane or thiazacyclo₄₋₇alkane, e.g., a compound of formula (VIII)

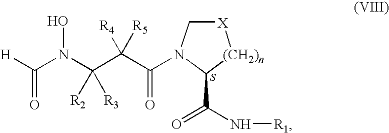

wherein R₁, R₂, R₃, R₄, R₅, X and n are as defined above.

To convert the compound of formula (VII) to the compound of formula (VIII), the hydroxy protecting group is removed using conventional hydrogenolysis techniques known in the art, e.g., by contacting the compound of formula (VII) with a palladium catalyst, such as Pd/BaSO₄ (see WO 02/102790 A1).

The R₁ moiety can be a heteroaryl, e.g., an azacyclo₄₋₇alkane, a thiazacyclo₄₋₇alkane or an imidazacyclo₄₋₇alkane. Specific examples of R₁ moieties in the compounds disclosed herein are heteroaryls of formula (X)

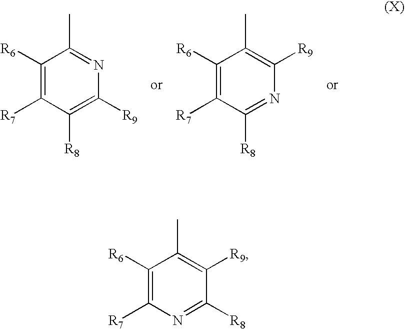

wherein each of R₆, R₇, R₈ and R₉, independently, is hydrogen, alkyl, substituted alkyl, hydroxy, alkoxy, acyl, acyloxy, SCN, halogen, cyano, nitro, thioalkoxy, phenyl, heteroalkylaryl, alkylsulfonyl or formyl.

A example of an R₁ moiety is a heteroaryl of formula (Xa)

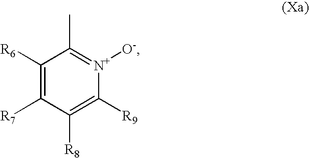

wherein R₆, R₇, R₈ and R₉ are as defined above for formula (X), e.g.,

wherein
a) R₆ is nitro, alkyl, substituted alkyl, phenyl, hydroxy, formyl, heteroalkylaryl, alkoxy, acyl or acyloxy; preferably alkyl, especially C₁₋₇alkyl; hydroxyl; or alkoxy, especially a C₁₋₇alkoxy; and
R₇, R₈ and R₉ are hydrogen; or
b) R₆, R₈ and R₉ are hydrogen; and
R₇ is alkyl, substituted alkyl, phenyl, halogen, alkoxy or cyano, preferably alkyl, especially C₁₋₇alkyl; substituted alkyl, especially substituted C₁₋₇alkyl, such as —CF₃; or alkoxy, especially C₁₋₇alkoxy; or
c) R₆, R₇ and R₉ are hydrogen; and
R₈ is alkyl, substituted alkyl, halogen, nitro, cyano, thioalkoxy, acyloxy, phenyl, alkylsulfonyl or carboxyalkyl, preferably alkyl, especially C₁₋₇alkyl; substituted alkyl, especially —CF₃; halogen; or carboxyalkyl; or
d) R₆, R₇ and R₈ are hydrogen; and
R₉ is alkyl, halogen or hydroxy; or
e) R₇ and R₉ are hydrogen; and
each of R₆ and R₈, independently, is halogen, alkyl, substituted alkyl, phenyl or cyano; or
f) Each of R₇ and R₉ is alkyl or substituted alkyl; and
R₆ and R₈ are hydrogen; or
g) R₆ and R₉ are hydrogen;
R₇ is alkyl or substituted alkyl; and
R₈ is nitro; or
h) R₈ and R₉ are hydrogen;
R₆ is cyano; and
R₇ is alkoxy; or
i) R₇ and R₈ are hydrogen; and
R₆ is alkyl, substituted alkyl, alkoxy or SCN; and
R₉ is alkyl or substituted alkyl; or
j) R₆ and R₇ are hydrogen;
R₈ is nitro or halogen; and
R₉ is alkyl or substituted alkyl; or
k) R₆, R₇, R₈ and R₉ are hydrogen; or
l) R₆ and R₇ together with the carbon atoms to which they are attached form a phenyl group, preferably substituted with hydroxy; and
R₈ and R₉ are hydrogen; or
m) R₆ and R₇ are hydrogen; and
R₈ and R₉ together with the carbon atoms to which they are attached form a phenyl group; or
n) n is 0; or
o) n is 0;
each of R₆, R₇, R₈ and R₉, independently, is hydrogen, alkyl or halogen; and
more particularly, R₆, R₇, R₈ and R₉ are hydrogen; or
p) n is 0;
R₆, R₈ and R₉ are hydrogen; and
R₇ is alkyl; or
q) n is 0;
R₆, R₇ and R₉ are hydrogen; and
R₈ is alkyl or halogen.

In another embodiment, R₁ is of formula (Xb)

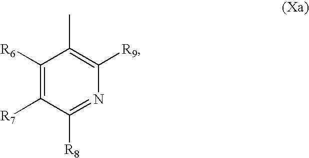

wherein
R₆, R₇, R₈ and R₉ are as defined above for formula (X); in particular, R₇ and R₈ together with the carbon atoms to which they are attached form a phenyl group; and
R₆ and R₉ are hydrogen.

In yet another embodiment, the R₁ is of formula (XI)

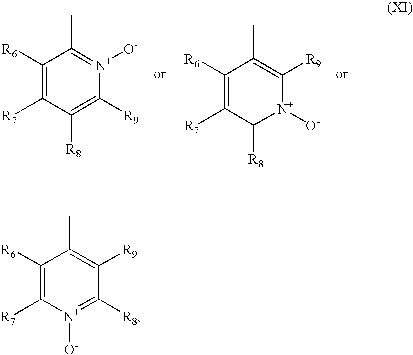

wherein each of R₆, R₇, R₈ and R₉, independently, is hydrogen, alkyl, substituted alkyl, phenyl, halogen, hydroxy or alkoxy, e.g.,

wherein
a) R₆ and R₈ are hydrogen;
R₉ is hydrogen or alkyl; and
R₇ is alkyl, substituted alkyl or phenyl; or
b) R₆, R₇ and R₉ are hydrogen; and
R₈ is halogen, alkyl or substituted alkyl; or
c) R₇, R₈ and R₉ are hydrogen; and
R₆ is hydroxy.

In a particularly useful embodiment the heteroaryl is of the formula (XIa)

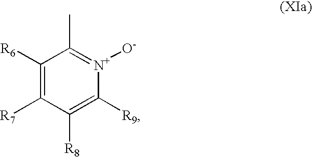

wherein R₆, R₇, R₈ and R₉ are as defined above for formula (XI).

In another embodiment, R₁ is an unsubstituted phenyl or the phenyl is substituted with alkoxy, e.g., methoxy; or aryloxy, e.g., phenoxy.

In another embodiment, the R₁ is of formula (XII)

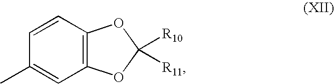

wherein each of R₁₀ and R₁₁, independently, is hydrogen or halogen. In particular, R₁₀ and R₁₁ are both either hydrogen or both halogen.

Unless otherwise stated, the following terms as used in the specification have the following meaning.

The term “cycloalkane” or “cycloalkyl” contains from 3- to 7-ring carbon atoms, and is, e.g., cyclopropyl, cyclobutyl, cyclopentyl and cyclohexyl.

The term “azacyclo₄₋₇alkane” contains 1-ring heteroatom which is a nitrogen. It contains from 4-7, and especially 4- or 5-ring atoms including the heteroatom.

The term “thiazacyclo₄₋₇alkane” contains 2-ring heteroatoms, nitrogen and sulfur. It contains from 4-7, and especially 5-ring atoms including the heteroatoms.

The term “imidazacyclo₄₋₇alkane” contains 2-ring heteroatoms which are both nitrogen. It contains from 4-7, and especially 5-ring atoms including the heteroatoms.

The term “alkyl” refers to saturated or unsaturated aliphatic groups, such as alkenyl or alkynyl, cycloalkyl or substituted alkyl including straight-chain, branched-chain and cyclic groups having from 1-10 carbons atoms. Preferably “alkyl” or “alk”, whenever it occurs, is a saturated aliphatic group or cycloalkyl, more preferably C₁₋₇alkyl, particularly C₁₋₄alkyl. Examples of “alkyl” or “alk” include, but are not limited to, methyl, ethyl, n-propyl, isopropyl, n-butyl, isobutyl, sec-butyl, t-butyl, n-pentyl, neopentyl, n-hexyl or n-heptyl, cyclopropyl and especially n-butyl.

The term “substituted alkyl” refers to an alkyl group that is substituted with one or more substituents preferably 1-3 substituents including, but not limited to, substituents, such as halogen, lower alkoxy, hydroxy, mercapto, carboxy, cycloalkyl, aryl, heteroaryl and the like. Examples of substituted alkyl groups include, but are not limited to, —CF₃, —CF₂—CF₃, hydroxymethyl, 1- or 2-hydroxyethyl, methoxymethyl, 1- or 2-ethoxyethyl, carboxymethyl, 1- or 2-carboxyethyl and the like.

The term “aryl” or “Ar” refers to an aromatic carbocyclic group of 6-14 carbon atoms having a single ring including, but not limited to, groups, such as phenyl; or multiple condensed rings including, but not limited to, groups, such as naphthyl or anthryl; and is especially phenyl.

The term “heteroaryl” or “HetAr” refers to a 4- to 7-membered, monocyclic aromatic heterocycle or a bicycle that is composed of a 4- to 7-membered, monocyclic aromatic heterocycle and a fused-on benzene ring. The heteroaryl has at least one hetero atom, preferably one or two heteroatoms including, but not limited to, heteroatoms, such as N, O and S, within the ring. A preferred heteroaryl group is pyridinyl, pyrimidinyl or benzdioxolanyl.

The aryl or heteroaryl may be unsubstituted or substituted by one or more substituents including, but not limited to, C₁₋₇alkyl, particularly C₁₋₄alkyl, such as methyl, hydroxy, alkoxy, acyl, acyloxy, SCN, halogen, cyano, nitro, thioalkoxy, phenyl, heteroalkylaryl, alkylsulfonyl and formyl.

The term “carbonylamine”, as used herein, refers to a —NHC(O)— group wherein the amino portion of the group is linked to the aryl/heteroaryl and the carbonyl portion of the group is linked to the azacyclo₄₋₇alkane, thiazacyclo₄₋₇alkane or imidazacyclo₄₋₇alkane.

The term “heteroalkyl” refers to saturated or unsaturated C₁₋₁₀alkyl as defined above, and especially C₁₋₄heteroalkyl which contain one or more heteroatoms, as part of the main, branched or cyclic chains in the group. Heteroatoms may independently be selected from the group consisting of —NR—, where R is hydrogen or alkyl, —S—, —O— and —P—; preferably —NR—, where R is hydrogen or alkyl; and/or —O—. Heteroalkyl groups may be attached to the remainder of the molecule either at a heteroatom (if a valence is available) or at a carbon atom. Examples of heteroalkyl groups include, but are not limited to, groups, such as —O—CH₃, —CH₂—O—CH₃, —CH₂—CH₂—O—CH₃, —S—CH₂—CH₂—CH₃, —CH₂—CH(CH₃)—S—CH₃ and —CH₂—CH₂—NH—CH₂—CH₂—.

The heteroalkyl group may be unsubstituted or substituted with one or more substituents, preferably 1-3 substituents including, but not limited to, alkyl, halogen, alkoxy, hydroxyl, mercapto, carboxy and, especially, phenyl. The heteroatom(s) as well as the carbon atoms of the group may be substituted. The heteroatom(s) may also be in oxidized form.

The term “alkoxy”, as used herein, refers to a C₁₋₁₀alkyl linked to an oxygen atom, or preferably C₁₋₇alkoxy, more preferably C₁₋₄alkoxy. Examples of alkoxy groups include, but are not limited to, groups, such as methoxy, ethoxy, n-butoxy, tert-butoxy and allyloxy.

The term “acyl”, as used herein, refers to the group —(O)CR, where R is alkyl, especially C₁₋₇alkyl, such as methyl. Examples of acyl groups include, but are not limited to, acetyl, propanoyl and butanoyl.

The term “acyloxy”, as used herein, refers to the group —OC(O)R, wherein R is hydrogen, alkyl, especially C₁₋₇alkyl, such as methyl or ethyl, or phenyl or substituted alkyl as defined above.

The term “alkoxycarbonyl”, as used herein, refers to the group —COOR, wherein R is alkyl, especially, C₁₋₇alkyl, such as methyl or ethyl.

The term “halogen” or “halo”, as used herein, refers to chlorine, bromine, fluorine, iodine and, is especially, fluorine.

The term “thioalkoxy”, as used herein, means a group —SR, where R is an alkyl as defined above, e.g., methylthio, ethylthio, propylthio, butylthio and the like.

The term “heteroalkylaryl”, as used herein, means a heteroalkyl group, e.g., —O—CH₂-substituted with an aryl group, especially, phenyl. The phenyl group itself may also be substituted with one or more substituents, such as halogen, especially, fluoro and chloro; and alkoxy, such as methoxy.

The term “alkylsulfonyl”, as used herein, means a group —SO₂R, wherein R is alkyl, especially, C₁₋₇alkyl, such as methyl sulfonyl.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups may be found in Greene et al., Protective Groups in Organic Synthesis, 3^{rd} Edition, John Wiley & Sons, Inc., NY (1999). Preferred hydroxy protecting groups include benzyl, Fmoc, TBDMS, photolabile protecting groups, such as Nvom, Mom and Mem. Other preferred protecting groups include NPEOC and NPEOM.

It will be appreciated that the compounds disclosed herein may exist in the form of optical isomers, racemates or diastereoisomers. In particular, in the compounds disclosed herein where R₄ and R₅ are different, the carbon atom to which the R₄ and R₅ groups are bonded is a chiral center and such compounds can exist in the R, S or racemic forms. It is contemplated that the process of the invention prepares the R optically pure form. By “optically pure” is meant that the enantiomeric purity is greater than 50%, preferably greater than 80%, more preferably greater than 90%, and most preferably greater than 95%. The optically pure R isomer of compound (I) can be used, in which case all subsequent compounds in the synthesis will remain in the R optically pure form, with respect to the same chiral carbon atom. Such R form of compound (I) is represented by former Ia below:

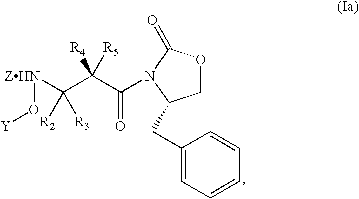

wherein R₂, R₃, R₄ and R₅ are as defined above. It is exemplified that in the compound of formula (I) that R₅ is hydrogen and that R₄ is C₂₋₁₀alkyl, more preferably C₂₋₇alkyl, and most preferably C₄alkyl.

It is further exemplified that in the optically pure compound of formula (I) that R₂, R₃, and R₅ are hydrogen and that R₄ is alkyl; such a compound has the structure (Ib)

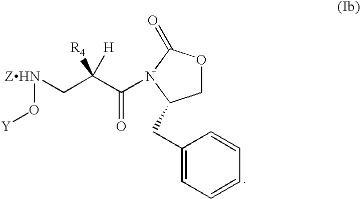

As an example, in compound (I), R₄ is n-butyl, where such compound has the structure (Ic)

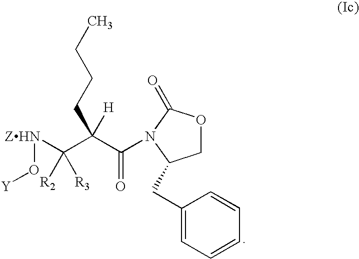

Further exemplified is that R₂, R₃ and R₅ are hydrogen and that R₄ is n-butyl; such compound has the structure (Id)

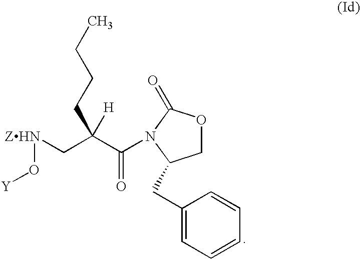

Alternatively, the racemate form of compound (I) can be used and then the R form can be resolved at a later step and the R form used for subsequent steps. For example, the compound formed after Step 3 or 3A, can be resolved into its RS and SS diastereomers and only the RS diastereomer used for subsequent steps. The RS diastereomer of compound (VII) is depicted below or formula (VIIa):

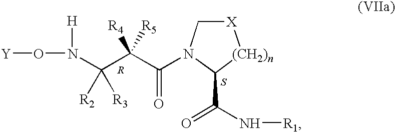

wherein R₂, R₃, R₄, R₅ Y, X, R₁ and n are as defined above, provided that R₄ and R₅ are different.

The optical isomers are resolved using standard techniques known in the art, for example, using silica gel column chromatography and an ethyl acetate/hexane solvent system. See, e.g., the methods taught in Chapter 4 of Advanced Organic Chemistry, 4^{th} Edition, March, John Wiley and Sons, NY (1992).

In the compounds disclosed herein, the following significances are exemplified individually or in any sub-combination:

1. R₁ is a heteroaryl of formula (Xa),

wherein
R₆, R₇ and R₉ are hydrogen and R₈ is methyl or trifluoromethyl; or
R₆, R₇ and R₉ are hydrogen and R₈ is fluoro; or
R₆, R₇ and R₈ are hydrogen and R₉ is fluoro; or
R₆, R₈ and R₉ are hydrogen and R₇ is ethyl or methoxy; or
R₇, R₈ and R₉ are hydrogen and R₆ is hydroxy; or
R₇ and R₈ are hydrogen, R₆ is methoxy and R₉ is methyl; or

R₁ is a heteroaryl of formula (Xb),
wherein
   R₆, R₇ and R₉ are hydrogen and R₈ is fluoro or trifluoromethyl; or
   R₆, R₈ and R₉ are hydrogen and R₇ is ethyl; preferably R₁ is a heteroaryl of formula (Xa),
   wherein R₆, R₈ and R₉ are hydrogen and R₇ is ethyl or a heteroaryl of formula (Xb),
   wherein R₆, R₇ and R₉ are hydrogen and R₈ is fluoro.
      2. X is —CH₂—, —CH(OH)—, —CH(OR)—, —CF₂— or —CH(F)—, preferably X is —CH₂—;
      3. R₄ is alkyl, preferably C₁₋₇alkyl, such as n-butyl;
      4. n is 1.

Temperature and pressure are not known to be critical for carrying out any of the steps of the invention, i.e., Steps 1A-4. Generally, for any of the steps, a temperature of about −10° C. to about 150° C., typically about 0° C. to about 80° C., is employed. Typically about atmospheric pressure is used for convenience; however, variations to atmospheric pressure are not known to be detrimental. Oxygen is not known to be detrimental to the process, therefore for convenience the various steps can be performed under ambient air, although an inert atmosphere, such as nitrogen or argon, can be used if desired. For convenience equimolar amounts of reactants or reagents, as appropriate, are typically used; however molar ratios can vary from about 1 to 2 equivalents, relative to the other reactant/reagent. The pH for the various steps is typically about 2 to about 12. The solvent used for the various steps are typically organic solvents, although in some situations aqueous/organic solvents can be used. Examples of suitable solvents include dioxane; methylene chloride; dichloromethane; toluene, acetone; methyl ethyl ketone; THF; isopropyl acetate; DMF; alcohols, especially, ethyl acetate, acetonitrile, higher-branched alcohols, such as t-butanol; and the like.

For Step 1A, a typical temperature is about 10° C. to about 40° C., more typically about 15° C. to about 25° C.; and a typical reaction time is about 0.1 hours to about 3 hours, more typically about 0.25 hours to about 1 hour. A pH of about 6 pH to about 10 pH, typically about 8 pH to about 9 pH, more typically about 9 pH, is employed. The base for Step 1A is a water soluble base such as sodium carbonate, sodium bicarbonate, potassium carbonate, potassium bicarbonate, an alkaline metal hydroxide, e.g., sodium hydroxide, potassium hydroxide, and the like. The solvent for Step 1A is a biphasic solvent, i.e., a mixture of water and an organic solvent immicible with water, for example, ethyl acetate, methylene chloride, diethyl ether, methyl t-butyl ether, isopropyl acetate, and the like. An example of a solvent is water/ethyl acetate. To prepare the starting compound of formula (I) for Step 1A (i.e., a salt) a strong acid is added to the corresponding free amine in solution with an organic solvent such as ethyl acetate, ethyl ether, and the like. The Z substituent, i.e., the strong acid, must be of sufficient strength to form a salt of the amine which results in the compound of formula (I) precipitating from the organic solution. The Z substituent is a strong organic or inorganic acid such as HCl, HBr, benzenesulfonic acid, toluenesulfonic acid, camphorsulfonic acid, and the like.

For Step 1B, a typical temperature is about −10° C. to about 10° C., more typically about −3° C. to about 2° C.; and a typical reaction time is about 0.5 hours to about 5 hours more typically about 0.75 hours to about 1.5 hours. The pH for Step B is typically about 8 pH to about 11 pH. The strong nucleophile/weak base used in Step 1B can be, for example, lithium hydroperoxide or a thiolate salt of an alkaline metal such as the sodium salt of propanethiol. The strong nucleophile/weak base is typically formed in situ, such as by adding hydrogen peroxide and an alkaline metal hydroxide, for example adding hydrogen peroxide and lithium peroxide to form lithium hydroperoxide in situ. The solvent for Step 2A can be a mixture of water and an ether solvent that is water miscible, such as THF, dimethylethane, dioxane, and the like. A typical solvent is THF/water.

For Step 2A, a typical temperature is about −20° C. to about 20° C., more typically about −10° C. to about 5° C.; and a typical reaction time is about 0.25 hours to about 2 hours, more typically about 0.3 hours to about 1 hour. The pH for Step 2A is typically, about 1 pH to about 6 pH. The formulating agent for Step 2A is typically formed in situ, such as by adding formic acid and acetic anhydride to form formic acetic anhydride. The solvent for Step 2A is an inert solvent in which the desired compound is soluble, for example, ethyl acetate, isopropyl acetate, methyl acetate, n-butyl acetate and the like. A typical solvent is ethyl acetate.

For Step 2B, a typical temperature is about −5° C. to about 40° C., more typically about 15° C. to about 25° C.; and a typical reaction time is about 1 hour to about 5 hours, more typically about 2 hours to about 3 hours. The pH for Step 2B is typically about 1 pH to about 6 pH. Typical solvents fro Step 2B include ethyl acetate, iso-propyl acetate, heptane, and the like. A particular example of a solvent is heptane. Examples of G substituents include —O^{⊖}metal^{⊕} wherein the metal is Na, K, Mg, Li, or —OH.amine wherein the amine of the formula HNR′R′, wherein each R′ is a straight chain, branched chain or cyclo alkyl group of 1 to 8 carbon atoms, more typically 1 to 6 carbon atoms. A typical example of a G substituent is —OH.amine wherein the amine is dicyclohexylamine. Therefore, an example of the compound of formula (V) has the structure:

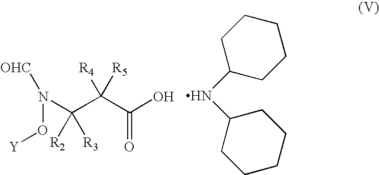

For Step 3, a typical temperature is about 10° C. to about 40° C., more typically about 15° C. to about 25° C.; and a typical reaction time is about 5 minutes to about 15 hours, more typically about 10 minutes to about 10 hours. The pH for Step 3 is typically about 5 to about 9. The solvent for Step 3 is a biphasic solvent, i.e., a mixture of water and an organic solvent immicible with water, for example, ethyl acetate, methylene chloride, diethyl ether, methyl t-butyl ether, isopropyl acetate, and the like. A typical solvent is water/ethyl acetate. Typical bases for Step 3 include tertiary amine bases such as N-methylmorphylene, triethyl amine, diisopropylethylamine, and the like. The coupling agent can be a conventional coupling agent known in the art, for example as disclosed in J. Jones, “The Chemical Synthesis of Peptides”, Clarendon, Oxford, 1991 ans P. Lloyd Williams, F. Albericio and E. Girault, Tetrahedron, 1993, 49, 11065, incorporated herein by reference. One or more coupling agents are used. Examples of coupling agents include EDCI, HOBt, DCC, HATU, BOP, FDPP, cross linked enzyme crystals such as PEPTI CLEC-TR, and the like. A typical coupling agent is EDCI/HOBt. A typical molar ration of DCCl:HOBt is about 1:5 to about 5:1.

For Step 4, a typical temperature is about 10° C. to about 35° C., more typically about 20° C. to about 22° C.; and a typical reaction time is about 60 minutes to about 18 hours, more typically about 4 hours to about 8 hours. The pH for Step 4 is typically about 4 to about 8. The solvent for Step 4 is typically an organic solvent, i.e., ethyl acetate, iso-propyl acetate, methylene chloride, and the like. The oxidizing agent can be a conventional agent known in the art, for example as disclosed in March, “Advanced Organic Chemistry”, 5th Ed., Wiley Interscience, NY, Chapter 19, incorporated herein by reference. Typical oxidizing agents include urea/hydrogen peroxide with phthalic anhydride; magnesium monoperoxyphthalate; MCPBA, Oxone (available from Aldrich), and the like.

Insofar as the production of starting materials is not particularly described, the compounds are known or may be prepared analogously to methods known in the art or as disclosed in the examples hereinafter.

The following abbreviations are used:
Ac=acetyl
BOP=[benzotriazol-1-yloxytris(dimethylamino)phosphonium hexafluorophosphate
CDMT=chlorodimethoxy triazine
DIEA=diisopropylethylamine
DCC=dicyclohexylcarbodimide
DMF=dimethylformamide
EDCI=1-(3-dimethylaminopropyl)-3-ethylcarbodiimide hydrochloride
2-EHA=2-ethylhexanoic acid
EtOAc=ethyl acetate
EtOH=ethanol
HATU=[O-(7-azabenzotriazol-1-yl)-N,N,N′,N′-tetramethyluronium hexafluorophosphate]isobutyl chloroformate
HPLC=high performance liquid chromatography
MCPBA=metachloroperoxybenzoic acid
MeOH=methanol
MMPP=magnesium monoperoxyphthalate
RT=room temperature
THF=tetrahydrofuran

The following illustrates a preferred process of the invention.

### Reaction Scheme

The following examples illustrate the invention but should not be interpreted as a limitation thereon. Product numbers refer to the preferred reaction scheme depicted immediately below.

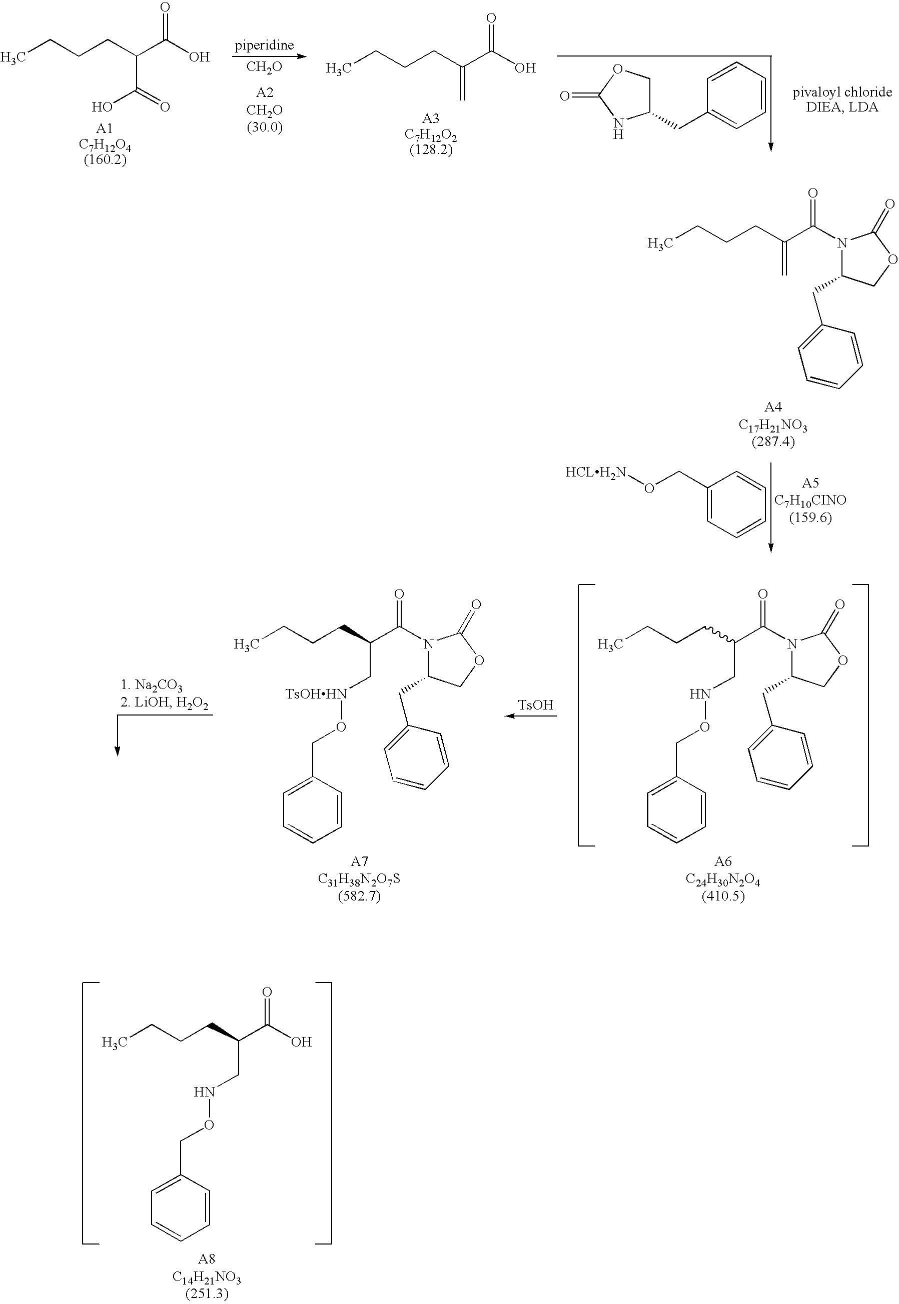

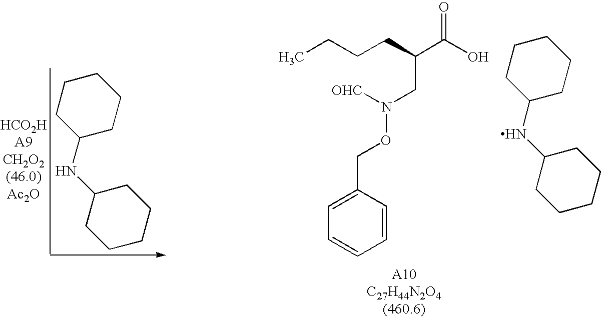

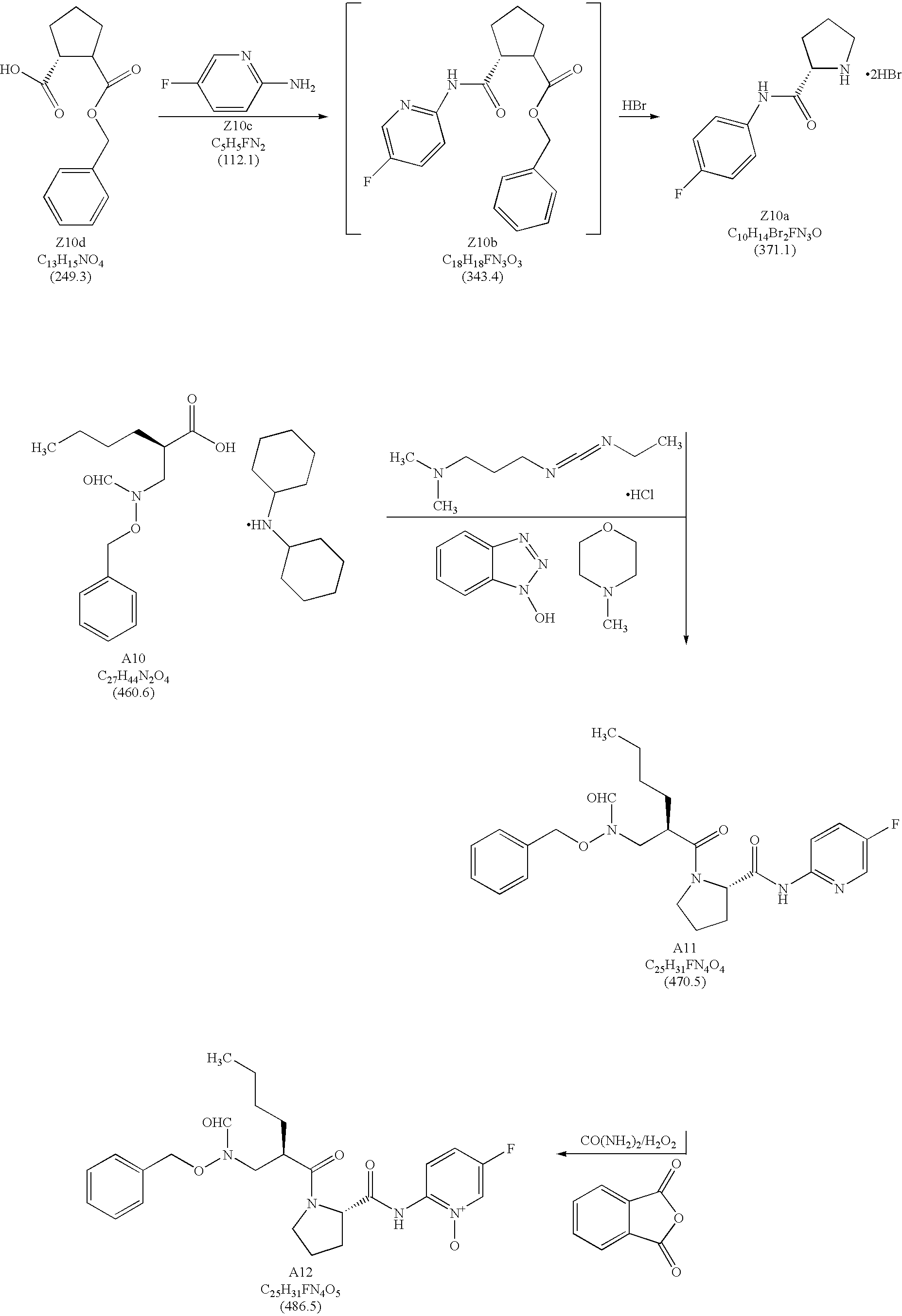

General procedure for the synthesis of intermediates useful for the preparation of:

### (2S)—N-(5-fluoro-1-oxido-2-pyridinyl-1-[(2R)-2-[formylhydroxyamino)methyl]-1-oxohexyl]-2-pyrrolidinecarboxamide, Magnesium Salt

### Step 1: (2R)-2-[[(phenylmethoxy)amino]methyl]-hexanoic Acid (A8)

To a solution of the p-TSA salt (A7) (58.3 g, 0.1 mol) in ethyl acetate (200 mL) and water (50 mL) was added 1 N Na₂CO₃ (185 mL). The two phase mixture was stirred for 15 minutes at RT and the lower aqueous layer was separated. The organic layer was washed with water (2×50 mL), and concentrated to give the free base of A7.

The A7 free base (41.0 g, 0.1 mol) was dissolved in THF (395 mL) and water (107 mL) and cooled to −3° C. To this solution was added 30% hydrogen peroxide (26.1 g, 0.23 mol) keeping the temperature at −3° C. In a separate flask, a solution of lithium hydroxide (5.0 g, 0.12 mol) in water (107 mL) was prepared and added slowly to the A7/hydrogen peroxide solution keeping the temperature at −3° C. The mixture was stirred for 45 minutes at this temperature.

A solution of sodium sulfite (43.5 g, 0.345 mol) in water (855 mL) was added slowly keeping the temperature below 10° C. and the reaction mixture was allowed to warm to RT. The solution was partially concentrated under vacuum to remove the THF and the aqueous portion was extracted with ethyl acetate (6×110 mL). The aqueous portion was then acidified with 3 N HCl (78 mL) and extracted with ethyl acetate (2×215 mL). The ethyl acetate extracts were combined and washed with water (2×110 mL). The organic solution was partially concentrated under vacuum (200 mL) to give a colorless solution of A8 which was used as is in the following step.

A sample was concentrated completely for characterization.

1H NMR (CDCl₃): δ 7.4 (s, 5H), 6.85 (bs, 2H), 4.75 (dd, 2H), 3.1 (m, 2H), 2.8 (m, 1H), 2.7 (m, 1H), 2.55 (m, 1H), 1.2 (m, 4H), 0.88 (m, 3H). ES-MS: calcd. for C₁₄H₂₁NO₃ (251.3); found: 252.2 [M+H].

### Step 2: (2R)-2-[[formyl(phenylmethoxy)amino]methyl]-hexanoic Acid Dicyclohexylamine Salt (A10)

Acetic Anhydride (15.3 G, 0.15 Mol) was Cooled to 0-5° C. and Treated with 96% Formic Acid (27.6 g, 0.6 mol) keeping the temperature below 10° C. The mixture was stirred for 15 minutes at 0-5° C. and then warmed to RT and stirred for 15 minutes more.

In a second flask, A8 ethyl acetate solution (502 g, 0.75 mol) was cooled to −15° C. and the formic acid/acetic anhydride mixture was added to it keeping the temperature at −10±5° C. The reaction mixture was stirred for 20 minutes at this temperature and then water (5.4 g) was added. After stirring for 15 minutes, the solution was warmed to RT. The solution was concentrated under vacuum (final volume=70-90 mL). Toluene (240 mL) was added and the solution was again concentrated under vacuum (final volume=70-80 mL).

In a separate flask a mixture of dicyclohexylamine (16.3 g) in heptane (240 mL) was prepared and this was added to the concentrate at RT. The mixture was seeded and held with stirring for 2 hours. Heptane (145 mL) was added and the suspension was held for 8 hours at RT. The solids were isolated by filtration and dried under vacuum to give the title compound.

m.p.: 83-86° C.; 1H NMR (CDCl₃, rotamers): δ 8.05 (bd, 1H), 7.3-7.65 (m, 5H), 4.75-5.1 (m, 2H), 3.5-4.0 (m, 2), 3.1-3.39 (m, 1H), 2.9 (m, 3H), 2.65 (m, 1H), 1.0-2.15 (m, 26H), 0.9 (s, 3H). ES-MS: calcd. for C₁₅H₂₁NO₄ (free acid) (279); found: 280.1 [M+H].

### Step 3: (2S)—N-(5-fluoro-2-pyridinyl)-1-[(2R)-2-[[formyl(phenylmethoxy)amino]methyl]-1-oxohexyl]-2-pyrrolidinecarboxamide (A11)

A solution of A10 (34.55 g, 75 mmol) in ethyl acetate (300 mL) was mixed with a citric acid solution (30 g of citric acid in 270 mL of water) and stirred at RT for 10 minutes. The layers were separated and the upper organic layer was washed with water (2×225 mL). At this point, N-(5-fluoro-2-pyridinyl)-(2S)-2-pyrrolidinecarboxamide dihydrobromide (33.39 g, 90 mmol) was added followed by water (60 mL) and HOBt (12.81 g, 82.5 mmol).

The mixture was cooled to 0-5° C. and EDCI (40.26 g, 210 mmol) and water (60 mL) were added. This was followed by the addition of N-methylmorpholine (47.79 g, 472.5 mmol). The reaction was stirred at RT overnight.

The lower aqueous layer was separated and the upper organic layer was washed with water (4×225 mL). The organic layer was filtered through a column of silica gel (83.4 g) and the column was further eluted with an additional volume of ethyl acetate (3×41 mL). The suitable fractions were combined and concentrated under vacuum to a specific volume (225 mL).

This solution was warmed to 50° C. and treated with heptane (675 mL). The solution was then cooled to 45° C. and seeded. The slurry was cooled to below −10° C. and held for 2 hours. The solids were isolated by filtration and dried under vacuum to give the title compound.

m.p.: 98° C.; 1H NMR (DMSO, rotamers): δ 10.6, 10.8 (s, 1H), 8.2 (s, 1H), 7.5-8.2 (m, 3H), 6.95-7.4 (m, 5H), 4.8 (s, 2H), 4.55 (bs, 1H), 3.2-3.8 (m, 4H), 2.9 (bs, 1H), 1.6-2.4 (m, 4H), 1.0-1.55 (m, 6H), 0.8 (s, 3H). ES-MS: calcd. for C₂₅H₃₁FN₄O₄ (470.6); found: 471.2 [M+H], 493.2 [M+Na].

### Step 4: (2S)—N-(5-fluoro-1-oxido-2-pyridinyl)-1-[(2R)-2-[[formyl(phenylmethoxy)amino]methyl]-1-oxohexyl]-2-pyrrolidinecarboxamide (A12)

A mixture of magnesium monoperoxyphthalate (69.25 g, 140 mmol) in water (128 mL) and isopropyl acetate (300 mL) was stirred and a solution of A11 (32.94 g, 70 mmol) in isopropyl acetate (162 mL) was added. The mixture was stirred for 17 hours at RT.

The bottom aqueous layer was separated and a solution of sodium sulfite (8.82 g, 70 mmol) in water (160 mL) was added. After stirring for 20 minutes, the bottom aqueous layer was separated and sodium carbonate (20 g, 190 mmol) in water (300 mL) was added. After stirring for 20 minutes, the bottom aqueous layer was separated and a solution of sodium chloride (19.0 g) in water (131 mL) was added. The layers were separated and the organic layer was concentrated under vacuum to a final volume of 92 mL.

The solution was filtered and the filtrate was heated to 40° C. and heptane (80 mL) was added. The solution was allowed to slowly cool to 30° C. and seed crystals were added. The mixture was held for one hour at this temperature and then cooled to 22° C. and more heptane was added (545 mL). After all of the heptane was added, the suspension was held at 22° C. for 2 hours and then further cooled to below −10° C. and held for 1 hour. The solids were isolated by filtration and dried under vacuum to give the title compound.

m.p.: 70° C.; 1H NMR (CDCl₃, rotamers): δ 10.35 (s, 1H), 8.45-8.75 (m, 1H), 7.61-8.45 (m, 2H), 7.35 (s, 5H), 7.05 (s, 1H), 4.65-5.22 (m, 2H), 4.1-4.65 (m, 1H), 3.25-4.1 (m, 3.5H), 2.64-3.2 (m, 1.5H), 1.02-2.42 (m, 10H), 0.85 (s, 3H). ES-MS: calcd. for C₂₅H₃₁FN₄O₅ (486.5); found: 487.2 [M+H].

What is claimed is:

1. A process for preparing a compound of the formula (VII) 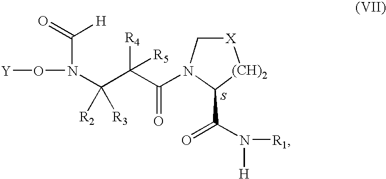
comprising Step 1A:
contacting a compound of the formula (I) 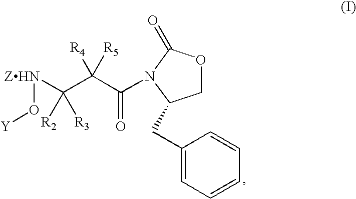
with a base in a suitable solvent to form the free base of compound (I), i.e., compound (II) of the formula (II) 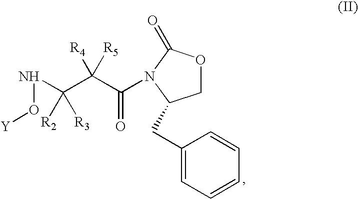
followed by Step 1B:
contacting compound (II) with a strong nucleophile/weak base in a suitable solvent under conditions to form compound (III) of the formula (III) 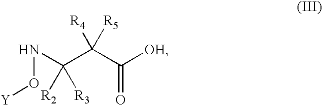
followed by Step 2A:
contacting compound (III) with a formulating agent in a suitable solvent under conditions suitable to form a compound of formula (IV) 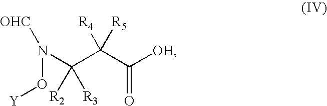
followed by Step 2B:
contacting compound (IV) with an amine or an alkaline metal hydroxide in a suitable solvent under conditions to form a compound of formula (V) 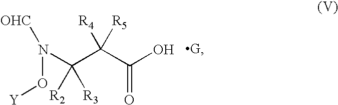
followed by Step 3:
contacting compound (V) with a compound of formula (VI) 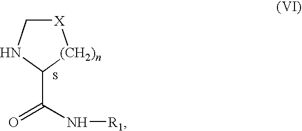
in the presence of a suitable base and one or more coupling agents in a suitable solvent under conditions to form a compound of formula (VII)
wherein
Y is a hydroxy protecting group;
each of R₂, R₃, R₄ and R₅ is, independently, hydrogen or alkyl, or (R₂ and R₃) and/or (R₄ and R₅) collectively form a C₄₋₇cycloalkyl;
G is —O^{⊖}metal^{⊕} or —OH.amine;
X is —CH₂—, —S—, —CH(OH)—, —CH(OR)—, —CH(SH)—, —CH(SR)—, —CF₂—, —C═N(OR)— or —CH(F)—;
R is alkyl;
R₁ is aryl or heteroaryl;
Z is a strong organic or inorganic acid; and
n is 0-3, provided that when n is 0, X is —CH₂—.

2. The process of claim 1 followed by Step 4, contacting the compound of formula VIII, wherein R₁ is heteroaryl having an N heteroatom, with an oxidizing agent to form the corresponding N-oxide derivative.

3. The process of claim 2 followed by the additional step of removing the hydroxyl protecting group of compound VII to form the compound of formula VIII: 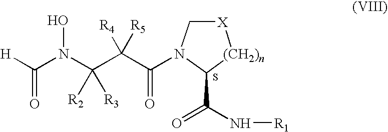
wherein R₁, R₂, R₃, R₄, R₅, X and n are as defined above.

4. The process of claim 1,
wherein
each of R₂, R₃ and R₅ is hydrogen;
R₄ is butyl;
X is —CH₂—;
n is 1;
Y is benzyl or t-butyldimethylsilyl; and
R₁ is of the formula 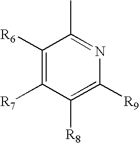
wherein
R₆ and R₉ are hydrogen;
R₇ is hydrogen or C₁₋₇alkyl; and
R₈ is hydrogen, halogen or C₁₋₇alkyl.

5. The process of claim 4,
wherein
R₇ is hydrogen; and
R₈ is fluoro.

6. The process of claim 1, wherein R₁ is of the formula (XIa) 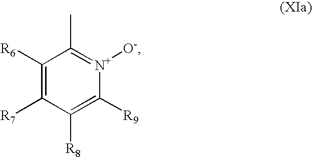
each of R₂, R₃ and R₅ is hydrogen;
RF is butyl;
X is —CH₂—;
n is 1;
Y is benzyl or t-butyldimethylsilyl;
R₆ and R₉ are hydrogen;
R₇ is hydrogen or C₁₋₇alkyl; and
R₈ is hydrogen, halogen or C₁₋₇alkyl.

7. The process of claim 6 wherein R₈ is halo or ethyl.

8. The process of claim 6 wherein R₇ is hydrogen and R₈ is fluoro.

9. The process of claim 1 wherein
for Step 1A the temperature is about 10° C. to about 40° C., the water soluble base is sodium carbonate, sodium bicarbonate, potassium carbonate, potassium bicarbonate, or an alkaline metal hydroxide, and the solvent is water/ethyl acetate, for Step 1B the temperature is about −10° C. to about 10° C., the strong nucleophile/weak base is lithium hydroperoxide, and the solvent is THF/water,
for Step 2A the temperature is about −20° C. to about 20° C., the formulating agent is formic acetic anhydride, and the solvent is ethyl acetate,
for Step 2B the temperature is about −5′ c to about 40° C., the solvent is heptane and the G substituent is of the formula —OH-amine wherein the amine is dicyclohexylamine,
for Step 3 the temperature is about 10° C. to about 40° C. th solvent is water/ethyl acetate, and the coupling agent is EDCI/HOBt, and
for Step 4 the temperature is about 10° C. to about 35° C., the solvent is ethyl acetate and the oxidizing agent is urea/hydrogen peroxide with phthalic anhydride or magnesium monoperoxyphthalate.

10. A process comprising
contacting a compound of the formula (I) 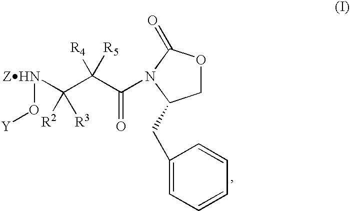
with a base in a suitable solvent to form compound (II) of formula 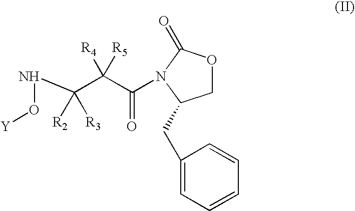
wherein
Y is a hydroxy protecting group;
each of R₂, R₃, R₄ and R₅ is, independently, hydrogen or alkyl, or (R₂ and R₃) and/or (R₄ and R₅) collectively form a C₄₋₇cycloalkyl;
and Z is a strong organic or inorganic acid.

11. A process comprising
contacting compound (II) of the formula 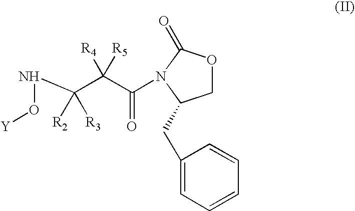
with a strong nucleophile/weak base in a suitable solvent under conditions to form compound (III) of the formula 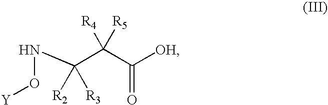
wherein
Y is a hydroxy protecting group; and
each of R₂, R₃, R₄ and R₅ is, independently, hydrogen or alkyl, or (R₂ and R₃) and/or (R₄ and R₅) collectively form a C₄₋₇cycloalkyl.

12. A process comprising
contacting compound (III) of the formula 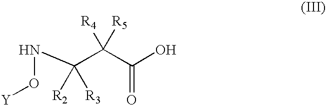
with a formulating agent in a suitable solvent under conditions suitable to form a compound of formula (IV) 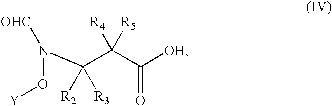
wherein
Y is a hydroxy protecting group; and
each of R₂, R₃, R₄ and R₅ is, independently, hydrogen or alkyl, or (R₂ and R₃) and/or (R₄ and R₅) collectively form a C₄₋₇cycloalkyl.

13. A process comprising
contacting compound (IV) of the formula 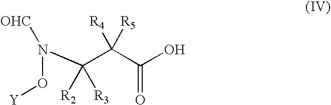
with an amine or an alkaline metal hydroxide in a suitable solvent under conditions to form a compound of formula (V) 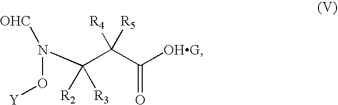
wherein
Y is a hydroxy protecting group;
each of R₂, R₃, R₄ and R₅ is, independently, hydrogen or alkyl, or (R₂ and R₃) and/or (R₄ and R₅) collectively form a C₄₋₇cycloalkyl; and
G is —O^{⊖}metal^{⊕} or —OH.amine.

14. A process comprising
contacting compound (V) of the formula 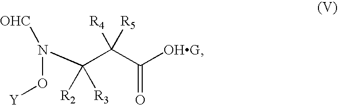
with a compound of formula (VI) 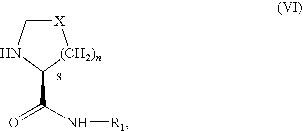
in the presence of a suitable base and one or more coupling agents in a suitable solvent under conditions to form a compound of formula (VII) 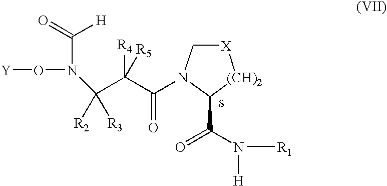
wherein
Y is a hydroxy protecting group;
each of R₂, R₃, R₄ and R₅ is, independently, hydrogen or alkyl, or (R₂ and R₃) and/or (R₄ and R₅) collectively form a C₄₋₇cycloalkyl;
G is —O^{⊖}metal^{⊕} or —OH.amine;
X is —CH₂, —S—, —CH(OH)—, —CH(OR)—, —CH(SH)—, —CH(SR)—, —CF₂—, —C═N(OR)— or —CH(F)—;
R is alkyl;
R₁ is aryl or heteroaryl; and
n is 0-3, provided that when n is 0, X is —CH₂.
